(19) United States
(12) Patent Application Publication
(10) Pub. No.: US 2005/0255048 A1
(43) Pub. Date: Nov. 17, 2005

(54) Sprayable formulations for the treatment of acute inflammatory skin conditions

(75) Inventors: Mark Hirsh, Wellesley, MA (US); Jane Hirsh, Wellesley, MA (US); Ira Skolnik, Acton, MA (US); Mark Trumbore, Westford, MA (US)

Correspondence Address: PATREA L. PABST;PABST PATENT GROUP LLP, 400 COLONY SQUARE, SUITE 1200, ATLANTA, GA, 30361, US

(73) Assignee: Collegium Pharmaceutical, Inc.

(21) Appl. No.: 11/128,947

(22) Filed: May 13, 2005

Related U.S. Application Data
(60) Provisional application No. 60/571,178, filed on May 15, 2004.
(60) Provisional application No. 60/655,306, filed on Feb. 23, 2005.

(51) Int. Cl. A61K 31/7048; A61K 31/704; A61K 31/496; A61K 31/4178; A61K 31/4196; A61K 31/58; A61K 31/65

(52) U.S. Cl. 424/44; 514/35; 514/152; 514/171; 514/254.07; 514/28; 514/383; 514/397

(57) ABSTRACT

A topical spray or foam, methods of making the formulation, and methods of use thereof, has been developed. In one preferred embodiment, the composition includes one or more active agents and exhibits both antibacterial activity and antifungal activity. Excipients such as chemical disinfectants, anti-pruritic agents to minimize itching, and skin protective compounds may be added. The composition may be formulated to be dispensed as a spray or foam and the spray or foam may be administered either by a hand pump or by an aerosolizing propellant. A second single phase formulation has also been developed. The formulation comprises a first drug which is water soluble or hydrophilic and a second drug which is lipid soluble or hydrophobic, wherein at least one of the drugs is bound to an ion-exchange resin. The use of binding resins, such as ion-exchange resins, allows drugs with incompatible solvent requirements to be prepared in a single-phase formulation.

### CROSS REFERENCE TO RELATED APPLICATIONS

This application claims benefit under 35 U.S.C. § 119 to U.S. Provisional Application Nos. 60/571,178, filed May 15, 2004; and 60/655,306, filed Feb. 23, 2005.

### FIELD OF THE INVENTION

This invention is generally in the field of formulations of antimicrobial and antifungal drugs for the treatment acute inflammatory skin conditions.

### BACKGROUND OF THE INVENTION

Skin is constantly exposed to the elements, making it susceptible to a variety of problems. Every year, more than 12 million people in the United States visit a doctor because of a skin rash, such as dermatitis. Dermatitis, also called eczema, is an inflammation of the skin. It can have many causes and occur in many forms. Generally, dermatitis describes swollen, reddened and itchy skin. A number of health conditions, allergies, genetic factors, physical and mental stress, and irritants can cause dermatitis.

Contact dermatitis results from direct contact with one of many irritants or allergens. Common irritants include laundry soap, skin soaps or detergents, and cleaning products. Possible allergens include rubber, metals such as nickel, jewelry, perfume, cosmetics, hair dyes, weeds such as poison ivy, and neomycin, a common ingredient in topical antibiotic creams. It takes a larger amount over a longer time for an irritant to cause dermatitis than it takes for an allergen: If one is sensitized to an allergen, just brief exposure to a small amount of it can cause dermatitis. Treatment consists primarily of identifying what's causing your irritation and then avoiding it. Sometimes, creams containing hydrocortisone or wet dressings that provide moisture to your skin may help relieve redness and itching. It can take as long as two to four weeks for this type of dermatitis to clear up.

Neurodermatitis can occur when something such as a tight garment rubs or scratches the skin. This irritation may lead one to rub or scratch the skin repeatedly. Common locations include ankles, wrist, outer forearm or arm, and the back of the neck. Hydrocortisone lotions and creams may help soothe the skin. Wet compresses may also provide relief. Sedatives and tranquilizers also may help stop scratching

Seborrheic dermatitis is often an inherited tendency, and is common in people with oily skin or hair. It may come and go depending on the season of the year. It may occur during times of stress or in people who have neurologic conditions such as Parkinson's disease. Commonly used shampoos contain tar, zinc pyrithione, salicylic acid or ketoconazole as the active ingredient. Hydrocortisone creams and lotions may soothe your skin and relieve itching. You also may need treatment for a secondary infection.

Stasis dermatitis can occur when fluid accumulates in the tissues just beneath the skin. The extra fluid initially thins out the skin and interferes with the blood's ability to nourish the skin. Wet dressings may be used to soften the thickened, yet fragile, skin and to control infection.

Atopic dermatitis often occurs with allergies and frequently runs in families in which other family members have asthma or hay fever. It usually begins in infancy and may vary in severity during childhood and adolescence. It tends to become less of a problem in adulthood, unless one is exposed to allergens or irritants in the workplace. Treatment typically consists of applying hydrocortisone-containing lotions to ease signs and symptoms. The newest treatment for this condition is a class of medications called immunomodulators, such as tacrolimus (Protopic) and pimecrolimus (Elidel). These medications affect the immune system and may help maintain normal skin texture and reduce flares of atopic dermatitis.

Diaper dermatitis, or diaper rash, is a broad term used to denote an acute inflammatory skin reaction in the “diaper area”, including the perineum, genitals, buttocks, lower abdomen, and inner thighs. It is the most common skin condition in infants, resulting in a large number of visits to physicians each year. The prevalence has been estimated at 35% to 75%, with peak incidence between 9 and 12 months.

Diaper rash is an even more serious problem among incontinent adults. It is estimated that there are over 10,000,000 affected adults in the United States. In nursing homes, the rate of some form of incontinence is estimated as 50% or more. Half of nursing home residents stay for long periods, averaging 19 months, and account for about 95% of nursing home days. In the year 2000, there were over 1.5 million people in nursing homes, about half of whom were over 85. Simply keeping residents clean is a major task in nursing home care, and in care of the elderly at home. Controlling or preventing acute skin inflammation is an ongoing concern. Moreover, the availability of medical consultation is often limited in a nursing home or home care environment, and consultation with specialists such as dermatologists is particularly difficult.

Clinically, diaper rash can be caused by local infection of the skin by either bacteria or fungi. In persistent inflammation, there can be colonization by both bacteria and fungi, and the combination can be especially damaging to skin, and difficult to treat. Such problems are common in cases of chronic incontinence in adults.

The severity of diaper dermatitis varies. Early signs include mild erythema, usually over a limited area, which may include minimal maceration and chafing of the skin. Moderate dermatitis is typified by marked erythema, often with papules, and usually includes maceration with or without satellite papules; it may cover a larger area and usually causes some pain and discomfort to the patient. In moderate dermatitis, C. albicans is frequently recovered from the rash and anal area.

Severe dermatitis is characterized by severe erythema with papulopustules over an extensive area. This may be accompanied by maceration of the affected area along with erosions and ulcerations, and patients experience marked pain. In this condition, both cleaning and application of ointments or creams typically is painful for the patient, and correspondingly difficult for the caregiver. At the same time, the treatment of the condition must rely primarily on non-medical caregivers.

Tinea is the general name given to skin infections caused by fungal dermatophytes. The most common human forms are tinea corposis (on the body); tinea captis or “ringworm” (on the head); tinea cruis, or “jock itch” (in the groin); and tinea pedis, or “athlete's foot” (on the feet). On the body and head, the initial fungal infection may spread outward from a focus as a ring (hence, “ringworm”), but generally does not permanently damage skin, and often clears up spontaneously. However, in the moist environment often found between the toes, tinea may persist and become a significant problem. It has been estimated that tinea pedis is the most common fungal infection in the world, affecting 30%-70% of the population.

There are two main anatomic forms of tinea pedis. One form is interdigital, which is also called intertriginous, which occurs between the toes. The interdigital form often is associated with puritis, erythema, scaling, and occasionally with fissures and maceration, particularly if there has been overgrowth with some bacterial or Candida species. The other form is plantar, which occurs on the sole or side of the foot. Within the plantar form, there are two distinctive types: “moccasin” and vesicobullous. The moccasin plantar type, which affects the sides of the foot, tends to be dry and scaling; sometimes there may be puritis; other times there may be some erythema. The vesicobullous type usually affects the plantar (ball) of the foot or the arch of the foot, and vesicles are the main component. There may be itching, scaling, and/or erythema. Most patients appear to have a combination of these symptoms, and it is rare to find a patient who has just one pure type. The term “athlete's foot” is a generic popular term, which is commonly used for any fungal infection of the foot; it is not a medical term.

Tinea pedis can cause complications if the patient is either immuno-suppressed or has any atopic condition; is diabetic; has compromised circulation; has undergone repeated trauma; has ill-fitting shoes or hammer toes; and/or is obese. Many of these factors are more likely to appear in the geriatric population. One complication may be cellulitis, or a spreading inflammation within solid tissue. Among people who have cellulitis of the lower extremities, a pre-existing tinea pedis infection has been found in a high percentage of these patients.

Although tinea pedis is usually considered to be a benign skin infection, acute and chronic web space tinea or dermatophytosis can predispose a patient to bacterial infections. The primary event in the pathogenesis is the invasion of the horny layer by dermatophytes. This infection appears as a mild to moderate scaly lesion and is asymptomatic. Dermatophytes are aerobic fungi that can cause infections of the skin, hair, and nails due to their ability to utilize keratin. The organisms colonize keratin-containing tissue and can cause fungal infections, e.g. tinea or ringworm, in association with the infected body part. The organisms are transmitted either by direct contact with infected hosts or by indirect contact with infected articles. Depending on the species the organism may be viable on an object for up to 15 months. The most common species of dermatophyte are Trichophyton rubum and Trichophyton metagrophytes. Trichophyton metagrophytes is responsible for about 15% of the cases and tends to be causative for the vesicular type; it may also spread to the nails. Epidermophyton floccosum tends to affect about 7% of the cases.

Leyden (J. J. Leyden & R. Aly, “Tinea Pedis”, Seminars in Dermatology, 12(4):280-284, (1993)) has proposed the term dermatophytosis simplex for the uncomplicated fungal type of scaling athlete's foot and dermatophytosis complex for the condition of macerated, itchy, often foul-smelling interspaces super-infected with bacteria. It is believed that asymptomatic cases of dermatophytosis simplex may progress to symptomatic dermatophytosis complex when the bacterial profile changes from a gram-positive bacterial ecosystem to a gram-negative bacterial over-growth.

As the gram-negative population increases, the recovery of dermatophytes in clinical samples decreases dramatically, and a point may be reached when no dermatophytes can be recovered from clinically symptomatic tinea pedis. Hence, clinically, the patient is diagnosed as having tinea pedis; but laboratory culture for fungus is negative. This former paradox is now identified and treated as gram-negative athlete's foot. Treatment of such cases to eliminate bacteria can create an opening for renewed infection by fungi, or by other bacteria. Moreover, systemic therapy for fungi is slow and requires high doses of anti-fungal medication, potentially causing side effects. What is needed is a treatment that can resolve simple cases, and that can maintain complex cases in a non-active state, or eliminate them entirely, once the major infective agent is identified and eliminated.

A further complication in treatments of tinea pedis and other tinea forms is the mode of administration. Conventionally, medications for external application for tinea are formulated as creams or ointments. Most patients can apply such ointments themselves, and so at least minimize cross-contamination with the fungi and bacteria. However, in institutional settings, and especially with handicapped patients, medication is often administered by others. In such cases, a conventional rub-in ointment is not optimal in isolating one patient from another, even with the use of gloves. Systemic administration of antifungal compounds is highly effective, but has an increased risk of side effects.

It is, therefore, an object of the invention to provide a composition comprising one or more active agents in an effective amount for treating inflammatory conditions of the skin, including in particular diaper rash and tinea pedis, which is easily administered.

### BRIEF SUMMARY OF THE INVENTION

A topical spray or foam, methods of making the formulation, and methods of use thereof, has been developed. In one preferred embodiment, the composition includes one or more active agents and exhibits both antibacterial activity and antifungal activity. Excipients such as chemical disinfectants, anti-pruritic agents to minimize itching, and skin protective compounds may be added. The composition may be formulated to be dispensed as a spray or foam and the spray or foam may be administered either by a hand pump or by an aerosolizing propellant.

A second single phase formulation has also been developed. The formulation comprises a first drug which is water soluble or hydrophilic and a second drug which is lipid soluble or hydrophobic, wherein at least one of the drugs is bound to an ion-exchange resin.

The use of binding resins, such as ion-exchange resins, allows drugs with incompatible solvent requirements to be prepared in a single-phase formulation.

### DETAILED DESCRIPTION OF THE INVENTION

I. Definitions

“Water Soluble” as used herein refers to substances that have a solubility of greater than or equal to 5 g /100 ml water.

“Lipid Soluble” as used herein refers to substances that have a solubility of greater than or equal to 5 g/100 ml in a hydrophobic liquid such as castor oil.

“Hydrophilic” as used herein refers to substances that have strongly polar groups that readily interact with water.

“Hydrophobic” as used herein refers to substances that lack an affinity for water; tending to repel and not absorb water as well as not dissolve in or mix with water.

“Resinate” as used herein refers to a drug reversibly bound to an ion exchange resin.

II. Composition

Two formulations have been developed. The first is a sprayable topical formulation or foam which has antibacterial and antifungal activities, where advantages are conferred through the combination of activities within a single spray or foam. In a second embodiment, the formulation comprises a first drug which is water soluble or hydrophilic and a second drug which is lipid soluble or hydrophobic, wherein at least one of the drugs is bound to an ion-exchange resin. The use of binding resins, such as ion-exchange resins, allows drugs with incompatible solvent requirements to be prepared in a single-phase formulation, which can be administered as a spray, foam, lotion, cream, ointment, or other type of topical preparation.

a. Excipients

Formulations may be prepared using pharmaceutically acceptable excipients composed of materials that are considered safe and effective and may be administered to an individual without causing undesirable biological side effects or unwanted interactions. The excipients are all components present in the pharmaceutical formulation other than the active ingredient or ingredients. As generally used herein “excipient” includes, but is not limited to, surfactants, emulsifiers, emulsion stabilizers, emollients, buffers, solvents and preservatives.

Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

Emollients

Suitable emollients include those generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethyihexyl palmitate.

Surfactants

Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Emulsifiers

Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Buffers

Buffers preferably buffer the composition from a pH of about 4 to a pH of about 7.5, more preferably from a pH of about 4 to a pH of about 7, and most preferably from a pH of about 5 to a pH of about 7.

Propellant

Preferred gaseous propellants for aerosol sprays or foams consist primarily of HFCs. Suitable propellants include HFCs such as 1,1,1,2-tetrafluoroethane (134a) and 1,1,1,2,3,3,3-heptafluoropropane (227), but mixtures and admixtures of these and other HFCs that are currently approved or may become approved for medical use are suitable. The propellants preferably exclude concentrations of hydrocarbon propellant gases, including particularly butanes, butenes, and propane, which are sufficient to produce flammable or explosive vapors during spraying. Furthermore, the aerosol spray has a limited concentration of volatile alcohols, including particularly ethanol, methanol, propanol and isopropanol, and butanols. The preferred limiting concentration in the mixture is, as with the gases, the concentration at which the sprayed material becomes flammable or explosive.

Drug Complexes

One or more of the active agents may be complexed with an ion-exchange resin. In one embodiment, the composition comprises a first drug, which is water soluble or hydrophilic and a second drug, which is lipid soluble or hydrophobic, wherein in at least one of the drugs is complexed to a binding resin. The complexes can be coated with resins or otherwise encapsulated to control or modify the rate and conditions of release of the drug into the body. The complexed drug is released from the ion-exchange resin in the presence of moisture. Such a complex is known as a “resinate”. The use of binding resins allows drugs with incompatible solvent requirements to be prepared in a single-phase formulation, which can exhibit greater stability, particularly at low temperatures.

An important class of binding resins is ion-exchange resins. Ion-exchange resins are water-insoluble materials, often cross-linked polymers, containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The matrix can also be inorganic, e.g., silica gel, or aluminosilicates, natively charged or modified by the addition of ionic groups, also referred to herein as “resins”.

The covalently bound salt forming groups may be strongly acidic (e.g., sulfonic or or sulfate acid groups), weakly acidic (e.g., carboxylic acid), strongly basic (e.g., quaternary ammonium), weakly basic (e.g., primary amine), or a combination of acidic and basic groups. Other types of charged groups can also be used, including any organic group that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group.

In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). The ion-exchange resins typically have exchange capacities below about 6 meq./g (i.e., 1 ionic group per 166 daltons of resin) and preferably below about 5.5 meq./g.

Resins suitable for use as described herein include many commercially available ion exchange resins such as “Dowex” resins and others made by Dow Chemical; “Amberlyte”, “Amberlyst” and other resins made by Rohm and Haas; “Indion” resins made by Ion Exchange, Ltd. (India), “Diaion” resins by Mitsubishi; BioRex Type AG and other resins by BioRad; “Sephadex” and “Sepharose” made by Amersham; resins by Lewatit, sold by Fluka; “Toyopearl” resins by Toyo Soda; “IONAC” and “Whatman” resins, sold by VWR; and “BakerBond” resins sold by J T Baker.

Preferred ion exchange resins will be those supplied in grades known to be suitable for, and approvable in, delivery of pharmaceuticals. Particular resins believed to be useful and approved include, without limitation, Amberlite IRP-69 (Rohm and Haas), and INDION 224, INDION 244, and INDION 254 (Ion Exchange (India) Ltd.). These resins are sulfonated polymers composed of polystyrene cross-linked with divinylbenzene.

The size of the ion-exchange particles should be less than about 2 millimeters, more preferably below about 1000 micron, more preferably below about 500 micron, and most preferably below about 150 micron (about 40 standard mesh). Commercially available ion-exchange resins (including Amberlite IRP-69, INDION 244 and INDION 254 and numerous other products) are typically available in several particle size ranges, and many have an available particle size range less than 150 microns. The particle size is not usually a critical variable in terms of drug release, but large particles give a formulation a “gritty” feel, which is not preferred when avoidable. When a formulation is to be sprayed, particle sizes below 100 microns, preferably below 50 microns, and most preferably even smaller, are preferred.

As used herein, the term “regularly shaped particles” refer to those particles which substantially conform to geometric shapes such as spherical, elliptical, and cylindrical. As used herein, the term “irregularly shaped particles” refers to particles excluded from the above definition, such as those particles with amorphous shapes with increased surface areas due to channels or distortions. For example, irregularly shaped ion-exchange resins of this type are exemplified by Amberlite IRP-69 (supplied by Rohm and Haas), and to the drug-resin complexes formed by binding drugs to these resins. Irregularly or regularly shaped particles may be used. The distinction between regularly shaped and irregularly shaped particles has been found by Kelleher et al (U.S. Pat. No. 4,996,047) to affect the degree of drug loading required to prevent swelling and rupture of coating when loaded resins are placed in salt solutions, in the absence of fillers or impregnating agents, such as polyethylene glycol. They found that the critical value was at least 38% drug (by weight in the drug/resin complex) in irregular resins, and at least 30% by weight in regular resins.

Ion exchange resins have pores of various sizes, which expand the area available for drug binding. The typical pore diameter is in the range of about 30 to 300 nanometers (nm), which is large enough for access by small-molecule drugs. For large drugs, such as proteins or nucleic acids, resins with larger pores, such as 500 to 2000 nm (0.5 to 2 micron), often called “macroreticular” or “macroporous”, are preferred.

Binding of drug to a charged (ion-exchange) resin can be accomplished according to any of four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). Other pharmaceutically acceptable cations, especially K and Li, can be substituted for Na. All of these reactions except (d) have cationic by-products and these by-products, by competing with the cationic drug for binding sites on the resin, reduce the amount of drug bound at equilibrium. For basic drugs, stoichiometric binding of drug to resin, i.e., binding an applied drug molecule to essentially each binding site while having a very low level of drug left in solution, is accomplished only through reaction (d).

Four analogous binding reactions can be carried out for binding an acidic drug to an anion exchange resin. These are: (a) resin (Cl-form) plus drug (salt form); (b) resin (Cl-form) plus drug (as free acid); (c) resin (as free base) plus drug (salt form); and (d) resin (as free base) plus drug (as free acid). Other pharmaceutically acceptable anions, especially Br, acetate, lactate and sulfate, can be substituted for Cl. All of these reactions except (d) have ionic by-products and the anions generated when the reactions occur compete with the anionic drug for binding sites on the resin with the result that reduced levels of drug are bound at equilibrium. For acidic drugs, stoichiometric binding of drug to resin (as above) is accomplished only through reaction (d).

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778, 4,894,239, and 4,996,047.

The result of treating the ion exchange resin with a solution of drug is a drug-loaded particle with no coating. Such a particle can be used for drug delivery with no additional treatment. However, the loaded particles will typically be coated with one or more layers of materials to control the rate and location of release of drug from the resin when a salt-containing aqueous solution is encountered.

b. Bioactive Ingredients

Bioactive agents include therapeutic, prophylactic and diagnostic agents. These may be organic or inorganic molecules, proteins, peptides, sugars, polysaccharides, or nucleic acid molecules. Examples of therapeutic agents include proteins, such as hormones, antigens, and growth effector molecules; nucleic acids, such as antisense molecules; and small organic or inorganic molecules such as antimicrobials, immunomodulators, decongestants, neuroactive agents, anesthetics, and sedatives. Examples of diagnostic agents include radioactive isotopes and radiopaque agents. The compositions can include more than one active agent.

Anti-Fungal Agents

A variety of known antifungal agents can be used to prepare the described composition. A list of potential anti-fungal agents can be found in “Martindale—The Complete Drug Reference”, 32nd Ed., Kathleen Parfitt, (1999) on pages 367-389. Suitable antifungals include, without limitation, amphotericin, amorolfine, bifonazole, bromochlorosalicyanilide, buclosamide, butenafine, butoconazole, candicidin, chlordantoin, chlormidazole, chlorphenesin, chlorxylenol, ciclopirox olamine, cilofungin, clotrimazole, croconazole, eberconazole, econazole, enilconazole, fenticlor, fenticonazole, fluconazole, flucytosine, griseofulvin, hachimycin, haloprogin, hydroxystilbamine, isethionate, iodochlorohydroxyquinone, isoconazole, itraconazole, ketoconazole, lanoconazole, luflucarban, mepartricin, miconazole, naftifine, natamycin, neticonazole, nifuroxime, nystatin, omoconazole, oxiconazole, pentamycin, propionic acid, protiofate, pyrrolnitrin, ravuconazole, saperconazole, selenium sulfide, sertaconazole, sulbentine, sulconazole, terbinafine, terconazole, tioconazole, tolciclate, tolnaftate, triacetin, timidazole, undecenoic acid, voriconazole and combinations thereof. Some of these agents are known to have antibacterial activity as well.

In a preferred embodiment, the anti-fungal agent(s) is an azole. Suitable imidazole and triazole antifungal agents are fluconazole, timidazole, secnidazole, miconazole nitrate, econazole, haloprogin, metronidazole, itraconazole, terconazole, posaconazole, ravuconazole, ketoconazole, clotimazole, sapirconazole and combinations thereof.

In an alternative embodiment, the anti-fungal agent(s) is chlorxylenol, undecyclenic acid, selenium sulfide, iodochlorohydroxyquinone, bromochlorosalicyanilide, triacetin or combinations thereof.

Preferred antifungal agents capable of being complexed to an ion-exchange resin include amorolfine, bensuldazic acid, benzoic acid, biphenamine, butenafine, butoconazole, chlormidazole, ciclopirox, cloconazole, clotrimazole, cloxyquin, dermostatin, econazole, halethazole, isoconazole, miconazole, monensin, naftifine, omoconazole, oxiconazole, nitrate, pecilocin, pyrithione, rubijervine, sertaconazole, sulconazole, terbinafine, ticonazole, and undecylinic acid.

Antibacterial Agents

A variety of known antibacterial agents can be used to prepare the described composition. A list of potential antibacterial agents can be found in “Martindale—The Complete Drug Reference”, 32nd Ed., Kathleen Parfitt, (1999) on pages 112-270. Classes of useful antibacterials include aminoglycosides, antimycobacterials, cephalosporins and beta-lactams, chloramphenicols, glycopeptides, lincosamides, macrolides, penicillins, quinolones, sulphonamides and diaminopyridines, tetracyclines, and miscellaneous. In a preferred embodiment, the antibacterial agent is selected from the group consisting of metronidazole, timidazole, secnidazole, erythromycin, bactoban, mupirocin, neomycin, bacitracin, cicloprox, fluoriquinolones, ofloxacin, cephalexin, dicloxacillin, minocycline, rifampin, famciclovir, clindamycin, tetracycline and gentamycin.

Suitable aminoglycosides include antibiotics derived from Streptomyces and other actinomycetales, including streptomycin, framycetin, kanamycin, neomycin, paramomycin, and tobramycin, as well as gentamycin, sissomycin, netilmycin, isepamicin, and micronomycin.

Suitable antimycobacterials include rifamycin, rifaximin, rifampicin, rifabutinisoniazid, pyrazinamide, ethambutol, streptomycin, thiacetazone, aminosalicylic acid, capreomycin, cycloserine, dapsone, clofazimine, ethionamide, prothionamide, ofloxacin, and minocycline.

Cephalosporins and beta-lactams generally have activity against gram-positive bacteria and newer generations of compounds have activity against gram-negative bacteria as well. Suitable cephalosporins and beta-lactams include:

First generation; cephalothin, cephazolin, cephradine, cephaloridine, cefroxadine, cephadroxil, cefatrizine, cephalexin, pivcephalexin, cefaclor, and cefprozil.

Second generation; cephamandole, cefuroxime axetil, cefonicid, ceforanide, cefotiam, and cephamycin.

Third generation; cefotaxime, cefmenoxime, cefodizime, ceftizoxime, ceftriaxone, cefixime, cefdinir, cefetamet, cefpodoxime, ceftibuten, latamoxef, ceftazidime, cefoperazone, cefpiramide, and cefsulodin.

Fourth generation: cefepime and cefpirome

Other cephalosporins include cefoxitim, cefmetazole, cefotetan, cefbuperazone, cefminox, imipenem, meropenem, aztreonam, carumonam, and loracarbef.

Chloramphenicols inhibit gram positive and gram negative bacteria. Suitable cloramphenicols include chloramphenicol, its sodium succinate derivative, thiamphenicol, and azidamfenicol.

Suitable glycopeptides include vancomycin, teicoplanin, and ramoplanin. Suitable lincosamides include lincomycin and clindamycin, which are used to treat primarily aerobic infections.

Macrolides have a lactam ring to which sugars are attached. Suitable macrolides include erytjhromycin, as well as spiromycin, oleandomycin, josamycin, kitamycin, midecamycin, rokitamycin, azithromycin, clarithromycin, dirithromycin, roxithromycin, flurithromycin, tylosin; and streptgramins (or synergistins) including pristinamycin, and virginiamycin; and combinations thereof.

Suitable penicillins include natural penicillin and the semisynthetic penicillins F, G, X, K, and V. Newer penicillins include phenethicillin, propicillin, methicilin, cloxacillin, dicloxacillin, flucloxacillin, oxacillin, nafcillin, ampicillin, amoxicillin, bacampicillin, hetacillin, metampicillin, pivampicillin, carbenecillin, carfecillin, carindacillin, sulbenecillin, ticarcillin, azlocillin, mezlocillin, piperacillin, temocillin, mecillinam, and pivemecillinam. Lactamase inhibitors such as clavulanic acid, sulbactam, and tazobacytam are often co-administered.

Suitable quinolones include nalidixic acid, oxolinic acid, cinoxacin, acrosoxacin, pipemedic acid, and the fluoroquinolones flumequine, ciprofloxacin, enoxacin, fleroxacin, grepafloxacin, levofloxacin, lomefloxacin, nadifloxacin, norfloxacin, ofloxacin, pefloxacin, rufloxacin, sparfloxacin, trovafloxacin, danofloxacin, enrofloxacin, and marbofloxacin.

Sulphonamides and diaminopyridines include the original of the “sulfa” drugs, sulphanilamide, and a large number of derivatives, including sulfapyridine, sulfadiazine, sulfafurazole, sulfamethoxazole, sulfadimethoxine, sulfadimethoxydiazine, sulfadoxine, sulfametopyrazine, silver sulfadiazine, mafenide acetate, and sulfasalizine, as well as related compounds including trimethoprim, baquiloprim, brodimoprim, ormetoprim, tetroxoprim, and in combinations with other drugs such as co-trimoxazole.

Tetracyclines are typically broad-spectrum and include the natural products chlortetracycline, oxytetracycline, tetracycline, demeclocycline, and semisynthetic methacycline, doxycycline, and minocycline.

Suitable antibacterial agents that do not fit into one of the categories above include spectinomycin, mupirocin, newmycin, fosfomycin, fusidic acid, polymixins, colistin, bacitracin, gramicidin, tyrothricin, clioquinol, chloroquinaldol, haloquinal, nitrofurantonin, nitroimidazoles (including metronizole, timidazole and secnidazole), and hexamine.

The antibiotic and antifungal agents may be present as the free acid or free base, a pharmaceutically acceptable salt, or as a labile conjugate with an ester or other readily hydrolysable group, which are suitable for complexing with the ion-exchange resin to produce the resinate.

Antiseptic Agents

Antiseptic agents can be included in compositions formulated for topical administration. Suitable antiseptic agents include iodine, iodophores including cadexomer iodine, chlorhexidine, gluconate, thimerosal, hydrogen peroxide, and peroxides and perchlorates including organic peroxides and perchlorate salts.

Skin Protectants

Skin protectants can be included in compositions formulated for topical administration. Such agents not only soothe the site of infection but may also aide in maintaining the integrity of the skin to prevent additional damage. Suitable skin protectants include allantoin; cocoa butter; dimethicone; kaolin; shark liver oil; petrolatum; lanolin; vegetable oils; ethoxylated oils and lipids; polymers such as polyalkylene oxides, polyvinylpyrrolidone, polyvinyl alcohol, poly(meth)acrylates, ethylvinyl acetate, polyalkylene glycols; polysaccharides and modified polysaccharides such as hyaluronic acid, cellulose ehers, cellulose esters, hydroxypropyl methylcellulose, crosscarmelose, and starch; natural gums and resins which may be gelling or non-gelling such as alginates, carrageenans, agars, pectins, glucomannans (guar, locust bean, etc.), galactomannans (e.g. konjac), gum arabic, gum traganth, xanthan, schleroglucan and shellac; and colloidal insolubles such as zinc oxide and other insoluble zinc salts, talcum powder and other micronized natural minerals; and colloidal silicas, aluminas and other metal oxides.

Local Anesthetics or Antihistamines

Local anesthetics or antihistamines may also be employed in the topical formulation in order to lessen the pain and itching caused by the local infection. Suitable local anesthetics and antihistamines include benzocaine, lidocaine, dibucaine, etidocaine, benzyl alcohol, camphor, resorcinol, menthol, and diphenhdramine hydrochloride.

III. Method of Making the Composition

The antibiotic-antifungal formulation is in the form of a spray or foam. The water in oil topical compositions may be in the form of emulsions such as creams, lotions, ointments, powders, micro emulsions, liposomes, or in the form of gels, liquids, aerosol spray, and aerosol foams (rigid foams). They may also be presented in dry powder formulations.

a. Emulsions

Emulsion Concentrate

The oil phase is prepared by mixing together the surfactant(s) and emulsifier(s) to melt. The aqueous phase is prepared separately by dissolving the preservatives in water with heating. The aqueous phase is added to the oil phase with continuous high shear mixing to produce a milky emulsion. The emulsion is cooled and the pH is adjusted by the addition of a buffer.

Separately, the active agent is suspended in a material such as propylene glycol and treated to eliminate any large aggregates. In a small scale operation, the mixture can be milled. The final active agent particle size is small enough to allow aerosolization, for example, less than about 20 microns in diameter, preferably less than about 10 microns, more preferably, less than about 5 microns. The active agent suspension is added to the emulsion with mixing. The formulation is brought to the final weight by the addition of water.

The concentration of the surfactant(s) in the concentrate is from about 0.5 to about 15% by weight of the final composition. The concentration of the emulsifier(s) is from about 0.5% to about 25% by weight of the final composition. The concentration of the buffer(s) is from about 1% to about 5% by weight of the final composition and the concentration of the stabilizer(s) is from about 5% to about 15% by weight of the final composition.

The composition of the active agent is about 0.01% to about 30% by weight of the final composition. The concentration of topical anesthetics is from about 1% to about 10% by weight and the concentration anti-fungals and other antibiotics is from about 0.3% to about 5% by weight.

Emulsion Formulation

The emulsion concentrate is placed in pressure cans, preferably coated aluminum cans to prevent corrosion, such as epoxy-coated cans. The lid and dispensing apparatus are crimped in place. The can is charged with propellant to the desired level. At the time of application, the mixture of the emulsion with the propellant may be insured by shaking, optionally with the aid of a mixing bead. The dispenser may be metered or unmetered (continuous). Metered dispensing is preferred for highly active materials. The less expensive continuous dispensing is preferred for non-critically measured active agents. The can may be arranged for either “upside down” spraying with the valve at the bottom, or the can have a dip tube so that the foam can be sprayed while the can is upright with the valve at the top. The concentration of the HFC propellant(s) is from about 10% to about 60% by weight of the final composition, more preferably about 20% to about 50% by weight of the final composition. In a preferred embodiment, the emulsion concentrate is mixed with an HFC propellant so that the final formulation in an aerosol can comprises about 50% to about 80% of concentrate and about 20% to about 50% of propellant. In a more preferred embodiment, the final formulation in an aerosol can contains 70% concentrate and 30% propellant.

b. Dry Powder Formulation

Ethanol and glycerin are mixed together to form a uniform solution. The pharmaceutically active agents are then dispersed in the solution to form a uniform mixture. Talc is suspended in the mixture and the suspension is placed into pressure cans, preferably coated aluminum cans to prevent corrosion. The lid and dispensing apparatus are then crimped into place and the can is charged with propellant to the desired level. At the time of the application, the talc and any other solids are suspended with shaking and the resulting suspension is dispensed in either a metered dose or unmetered dose.

C. Ion-Exchange Formulation

The ion exchange resin is slowly added to distilled water with stirring to form a slurry. The drug to be administered is added to the slurry containing the ion-exchange resin and the resulting mixture is stirred. The drug-resin complex is separated from the mother liquor using vacuum filtration. The drug-resin complex is washed several times with distilled water to remove uncomplexed drug and the complex is dried under vacuum. The complex is dried in a 45° C. oven until the residual moisture is less than 10%, i.e. until the weight loss upon drying of sample in a moisture balance is less than 10%.

IV. Method of Administering the Composition

a. Administration of the Formulation to a Patient

The composition can be formulated to be dispensed by spraying. The spray may be administered using a hand pump or by the use of an aerosolizing propellant. In an alternative embodiment, the spray formulation may form a film on the skin. In yet another embodiment, film formation may result from the evaporation of a non-aqueous solvent or of water from an applied fluid or foam.

A selected amount of product is dispensed from the spray can, preferably onto the site to be treated. For non-critical active agents, the foam can be administered into the palm of the hand (the latter is also preferred when the application site in not visible). The amount to be delivered can be determined by the prescribing physician or as directed in the instructions for non-prescription products. Alternatively, a fixed dose using the metering dispenser can be administered. The foam is rubbed into the skin at the site to be treated. Because the foam is stable at body temperature, this step does not need to be hurried. Moreover, the exact site of application can be more easily controlled. If contact with the hand is to be avoided, a glove may be worn; or, the foam may be left in place, wherein it will eventually collapse and deliver the active ingredient to the surface of the skin.

A spray powder is a suspension of a particulate material, such as talc, in a non-aqueous solution that is compatible with the skin. Typically, part of the solution is volatile. Spraying action is provided by either a simply pump, or more commonly a pressurized gas, such as an alkane or a hydrofluoroalkane. After the evaporation of volatile components, the active ingredients are partially on the skin and partially on the carrier, and have a silky and soothing feel.

Any of the foregoing can be provided in combination with a kit to clean the skin and enhance application and/or efficacy. For example, the kit can include one or more materials for cleansing of the area to be treated. The materials can in the form of a spray, lotion, cream, gel, aerosol spray.

The materials will have one of the following capabilities:

Moisturizing dermal wound cleaner which removes debris and exudate as it cleanses and washes.

Hydrogel that protects the wound from foreign contaminants.

Materials that do not dry out.

Eliminates odors.

Contains glycerin to moisturize skin.

Contains surfactant such as poloxomer which cleanses skin without drying out.

Astringent skin cleanser.

Abradent skin cleanser

Easy to apply and easy to remove.

The present invention will be further understood by reference to the following non-limiting examples.

### EXAMPLES

Examples 1-5 below are made by the following general methods:

1. The oil phase is prepared by mixing the emollient oils (mineral oil, etc.) and the emulsifiers and heating the mixture to 70-80° C.

2. The aqueous phase is prepared separately by mixing about 80% of the water and the glycerin together with stirring while heating to about 70-80° C.

3. The aqueous phase is then added to the oil phase with continuous high shear mixing to produce a milky emulsion.

4. The emulsion is then cooled to about 30-40° C.; the emulsion thickens but remains a liquid.

5. The pH is adjusted if necessary by the addition of triethanolamine.

6. Separately, the preservative is dissolved in the propylene glycol with stirring. Then, for Examples 1-3 and 5, the active ingredients clindamycin, metronidazole and optionally muciprocin, are suspended in propylene glycol and treated to eliminate any large aggregates. (In example 4, diphenylhydramine HCl, menthol and allantoin are the active ingredients.) In a small scale operation, the mixture is milled. The final active agent particle size is small enough to allow aerosolization, for example, less than about 20 microns in diameter, preferably less than about 10 microns, more preferably, less than about 5 microns.

7. The active agent suspension is added to the emulsion with mixing.

8. The formulation is brought to the final weight with the addition of water.

The amount of triethanolamine is sensitive to the particular lots of ingredients, and the amount added determines the final pH of the product. The preferred pH in this formulation is about pH 4 to about 7, which is generally provided by the proportion of TEA listed.

### Example 1

### Cream Containing One Antibacterial Agent and One Anti-Fungal Agent

| Ingredient | Weight % |
|---|---|
| Clindamycin | 1.0 |
| Metronidazole | 0.75 |
| Water | 67.65 |
| Propylene Glycol | 5.0 |
| Glycerin | 2.5 |
| Polyglyceryl-3 Methylglucose | 3.0 |
| Distrearate | |
| Ethylhexyl Stearate | 6.0 |
| Ethylhexyl Palmitate | 5.0 |
| Mineral Oil | 5.5 |
| Glyceryl Stearate | 1.8 |
| Stearyl Alcohol | 0.8 |
| Cetyl Dimeticone | 1.0 |
| Preservative | q.s. |

### Example 2

### Cream Containing Two Antibacterial Agents and One Anti-Fungal Agent

| Ingredient | Weight % |
|---|---|
| Clindamycin | 1.0 |
| Muciprocin | 2.0 |
| Metronidazole | 0.75 |
| Water | 65.65 |
| Propylene Glycol | 5.0 |
| Glycerin | 2.5 |
| Polyglyceryl-3 Methylglucose | 3.0 |
| Distrearate | |
| Ethylhexyl Stearate | 6.0 |
| Ethylhexyl Palmitate | 5.0 |
| Mineral Oil | 5.5 |
| Glyceryl Stearate | 1.8 |
| Stearyl Alcohol | 0.8 |
| Cetyl Dimeticone | 1.0 |
| Preservative | q.s. |

### Example 3

### Lotion Containing An Oil/Water Emulsion

| Ingredient | Weight % |
|---|---|
| Clindamycin | 1.0 |
| Metronidazole | 0.75 |
| Water | 83.3 |
| Methyl Glucose Sesquistearate | 2.0 |
| Glycerin | 3.0 |
| Ethylhexyl Stearate | 6.0 |
| Mineral Oil | 5.7 |
| 10% Sodium Hydroxide | q.s. |
| Preservative | q.s. |

### Example 4

### Cream with an Anti-Pruritic Agent, a Local Anesthetic and a Skin Protectant

| Ingredient | Weight % |
|---|---|
| Diphenhydramine hydrochloride | 1.0 |
| Menthol | 1.0 |
| Allantoin | 0.2 |
| Water | 67.05 |
| Propylene Glycol | 5.0 |
| Glycerin | 2.5 |
| Polyglyceryl-3 Methylglucose | 3.0 |
| Distrearate | |
| Ethylhexyl Stearate | 6.0 |
| Ethylhexyl Palmitate | 5.0 |
| Mineral Oil | 5.5 |
| Glyceryl Stearate | 1.8 |
| Stearyl Alcohol | 0.8 |
| Cetyl Dimeticone | 1.0 |
| Preservative | q.s. |

### Example 5

### Spray Foam Containing Two Antibacterial Agents and One Antifungal Agent

A. Concentrate

| Ingredient | Weight % |
|---|---|
| Clindamycin | 1.0 |
| Muciprocin | 2.0 |
| Metronidazole | 0.75 |
| Water | 30.95 |
| Propylene Glycol | 2.5 |
| Glycerin | 1.25 |
| Polyglyceryl-3 Methylglucose | 1.5 |
| Distrearate | |
| Ethylhexyl Stearate | 3.0 |
| Ethylhexyl Palmitate | 2.5 |
| Mineral Oil | 2.75 |
| Glyceryl Stearate | 0.9 |
| Stearyl Alcohol | 0.4 |
| Cetyl Dimeticone | 0.5 |
| Preservative | q.s. |
| Propellant HFC 134a | 50.0 |

B. Propellant

The concentrate is placed in an aerosol spray can, and the can is loaded with HFC134a so that the composition is approximately 70% concentrate and 30% HFC, i.e., 3 grams of propellant are added per 7 grams of concentrate.

### Example 6

### Spray Powder with Antibacterial and Antifungal Agents

Example 6 was made by the following method.
1. Ethanol and glycerin are mixed together to form a uniform solution.
2. The clindamycin, muciprocin and metronidazole are dissolved in the ethanol/glycerin solution.
3. Talc is then suspended in the mixture.
4. The suspension is added to aerosol cans.

5. The can is then loaded with HFC134a so that the final composition is approximately 50% suspension and 50% propellant.

| Ingredient | Weight % |
|---|---|
| Clindamycin | 1.0 |
| Muciprocin | 2.0 |
| Metronidazole | 0.75 |
| Ethanol | 35.25 |
| Talc | 10.0 |
| Glycerin | 1.0 |
| Propellant HFC 134a | 50.0 |

### Example 7

### Doxycycline Hyclate/Ion-Exchange Resin Complex Preparation

1. 100 g ion-exchange resin (Amberlyte IRP 69, Rohm & Haas) is slowly added to 800 ml distilled water while gently stirring; the slurry is stirred for an additional 15 minutes following complete addition of resin.

2. 106.6 g of doxylcycline hyclate is added to the slurry and stirred for 2 hours.

3. The drug-resin complex (“resinate”) is harvested by vacuum filtration using a Buchner funnel with a medium pore fritted disk.

4. The resinate was washed with 900 ml distilled water and vacuum dried as above to remove uncomplexed drug.

5. The resinate was washed a second time with 900 ml distilled water and vacuum dried.

6. The resinate was washed a third time with 900 ml distilled water and vacuum dried.

7. The washed resinate was dried in a 45° C. oven until the residual moisture was less than 10%, i.e., until the weight loss upon drying of a sample in a moisture balance was less than 10%.

### Example 8

### Non-Aerosol Spray Formulation Containing Ketoconazole and Doxycycline Resinate

| | Weight % | Mass Required 250 gram batch | |
|---|---|---|---|
| Active Ingredients | | | |
| Doxycycline Resinate | 10.00 | 25.000 | g |
| Ketoconazole | 2.00 | 5.000 | g |
| Inactive Ingredients | | | |
| Castor Oil | 84.75 | 211.875 | g |
| Polyoxy 10 Oleyl Ether | 2.00 | 5.000 | g |
| Fumed Silica | 1.25 | 3.125 | g |

1. Dissolve 5.00 g Polyoxy 10 Oleyl Ether in 211.875 g Castor Oil with gentle stirring.

2. Dissolve 5.00 g Ketoconazole in the Castor Oil/Surfactant solution with moderate heat and gentle stirring.

3. Cool to room temperature with constant gentle stirring.

4. Suspend 25.00 g of the Doxycycline Resinate (as prepared in Example 1) in the castor oil solution with moderate stirring.

5. Suspend 3.125 g Fumed Silica in the slurry of step 4 with moderate stirring.

6. Mix the slurry of step 5 under high shear until uniform and smooth.

7. Package in a spray bottle.

The spray bottle was obtained from a hardware store and is a plastic spray bottle with a trigger sprayer, intended for general household use. The preparation was used to dispense the preparation, which was clear except for the yellow-orange ion exchange resin particles. It was possible to dispense the preparation repeatedly without clogging the nozzle. The sprayed preparation had an oily feel with a slight grittiness from the resin.

### Example 9

### Non-Aerosol Spray Formulation Containing Ketoconazole, Zinc Oxide and Doxycycline Resinate

The preparation of Example 8 was repeated, with the inclusion of zinc oxide as a skin protective agent.

| | Weight % | Mass Required 250 gram batch | |
|---|---|---|---|
| Active Ingredients | | | |
| Doxycycline Resinate | 10.00 | 25.00 | g |
| Zinc Oxide | 10.00 | 25.00 | g |
| Ketoconazole | 2.00 | 5.00 | g |
| Inactive Ingredients | | | |
| Castor Oil | 75.5 | 188.75 | g |
| Polyoxy 10 Oleyl Ether | 2.00 | 5.00 | g |
| Fumed Silica | 1.25 | 1.25 | g |

1. Dissolve 5.00 g Polyoxy 10 Oleyl Ether in 188.75 g Castor Oil with gentle stirring.

2. Dissolve 5.00 g Ketoconazole in Castor Oil/Surfactant with moderate heat and gentle stirring.

3. Cool to room temperature with constant gentle stirring.

4. Suspend 25.00 g Doxycycline Resinate in solution with moderate stirring.

5. Suspend 25.00 g Zinc Oxide in slurry with moderate stirring

6. Suspend 1.25 g Fumed Silica in slurry with moderate stirring.

7. Mix slurry under High Shear until uniform and smooth.

8. Package in spray bottle.

The slurry was opaque, due to the zinc oxide, and tan-colored from the resin. It sprayed smoothly.

### Example 10

### Metronidazole/Terginafine Resinate Spray Powder Formulation

1. Ethanol and glycerin are mixed together to form a uniform solution.

2. Metronidazole is dissolved in the ethanol/glycerin solution.

3. Terbinafine resinate and talc are then suspended in the mixture.

4. The suspension is added to aerosol cans.

5. The can is then charged with HFC134a so that the final composition is approximately 50% suspension and 50% propellant.

| Ingredient | Weight % |
|---|---|
| Ethanol | 35.25 |
| Glycerin | 1.00 |
| Metronidazle | 0.75 |
| Terbinafine Resinate | 6.00 |
| Talc | 7.00 |
| HFC134a | 50.00 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A topical spray or foam formulation comprising one or more antifungal and antibacterial active agents in an effective amount to treat or reduce the symptoms associated with diseases or disorders of the skin in a pharmaceutically acceptable spray or foaming excipient.

2. The spray of claim 1 wherein the disease or disorder is selected from the group consisting of tinea pedis, diaper rash, contact dermatitis, neurodermatitis, seborrheic dermatitis, stasis dermatitis, and atopic dermatitis.

3. The formulation of claim 1 wherein the active agent is a single compound having antimicrobial and antifungal activity.

4. The formulation of claim 1 wherein the active agent has activity against gram negative and gram positive bacteria or dermatophytes.

5. The formulation of claim 1 comprising multiple antibacterial agents.

6. The formulation of claim 1 further comprising an ion-exchange resin.

7. The formulation of claim 1 wherein the pH of the formulation is in the range of about pH 3 to about pH 7.

8. The formulation of claim 1 further comprising an agent selected from the group consisting of antipruritic agents, skin protective agents, and antiseptic agents.

9. The formulation of claim 8 comprising an antipruritic agent selected from the group consisting of antihistamines, topical anesthetics, and combinations thereof.

10. The formulation of claim 8 comprising an antiseptic agent selected from the group consisting of iodine, iodophos, chlorhexidine, gluconate, thimerosol, hydrogen peroxide, benzoyl peroxide, metal salts and combinations thereof.

11. The formulation of claim 8 comprising a skin protective agent selected from the group consisting of allantoin, cocoa butter, dimethicone, kaolin, shark liver oil, petrolatum, lanolin, vegetable oils, ethoxylated oils and lipids, polyalkylene oxides, polyvinylpyrrolidone, polyvinyl alcohol, polysaccharides, water repellant insoluble colloidal materials, emollients, lubricants, occlusive moisturizers, metal oxides, metal salts, plasticizers, surfactants and combinations thereof.

12. The formulation of claim 11 wherein the excipient comprises volatile components and the skin protecting material forms a barrier after the evaporation of volatile components of the excipients.

13. The formulation of claim 12 wherein the barrier protects the skin from external liquid water for at least 3 hours.

14. The formulation of claim 1 wherein the excipient is sufficiently volatile at room temperature that it dries within about 1 minute under normal room conditions.

15. The formulation of claim 1 wherein the vehicle comprises at least one of volatile hydrocarbons and hydrofluorocarbons.

16. The formulation of claim 16 wherein the vehicle contains less than 5% of a volatile lower alcohol

17. The formulation of claim 1 wherein the antifungal component is selected from the group consisting of terbinafine, ciclopirox, nystatin, miconazole, nafitine, clotrimazole, ketoconazole, griseofulvin, fluconazole, voriconazole, oxiconazole, tolnafate, haloprogin, butoconazole, sertaconazole, terconazole, ticonazole, korostatin and echinocandins, and pharmaceutically acceptable salts, labile esters, ionic conjugates, and encapsulated forms thereof.

18. The formulation of claim 1 wherein the antibacterial component is selected from the group consisting of mupirocin, fusidic acid, paromomycin (neomycin E), doxycycline, and neomycin (neomycin A, B, C), and pharmaceutically acceptable salts, labile esters, ionic conjugates, and encapsulated forms thereof.

19. The formulation of claim 1 in a propellant-pressurized container.

20. The formulation of claim 1 in a hand-pumped non-pressurized container.

21. A method for the treatment of a patient in need thereof, comprising administering the formulation of any of claim 1 to a site thereon.

22. A single phase formulation comprising a first drug which is hydrophilic or water soluble, a second drug which is hydrophobic or lipid soluble, and an ion-exhange resin, wherein at least one of the drugs binds to the ion exchange resin.

23. The formulation of claim 22 wherein the first drug is bound to the ion-exchange resin.

24. The formulation of claim 22 wherein the second drug is bound to the ion-exchange resin.

25. The formulation of claim 22 further comprising an excipient for topical administration selected from the group consisting of lotions, creams, ointments, foams, sprays, gels, solutions, and suspensions.

26. The formulation of claim 25 wherein the drugs are selected from the group consisting of antibiotics and antifungals.
